(19) United States
(12) Patent Application Publication
(10) Pub. No.: US 2024/0369571 A1
(43) Pub. Date: Nov. 7, 2024

(54) STANDARDISATION OF NUCLEOSOME ASSAYS USING BIOLOGICALLY DERIVED CALIBRANTS

(71) Applicant: Belgian Volition SRL, Isnes (BE)

(72) Inventors: Jacob Vincent MICALLEF, London (GB); Mark Edward ECCLESTON, London (GB); Theresa K. KELLY, San Marcos, CA (US)

(21) Appl. No.: 18/568,228

(22) PCT Filed: Jun. 8, 2022
(86) PCT No.: PCT/EP2022/065558

§ 371 Date: Dec. 7, 2023

(30) Foreign Application Priority Data
Jun. 8, 2021 (GB) 2108185.6

(51) Int. Cl. G01N 33/68; G01N 33/96

(52) U.S. Cl. CPC G01N 33/6875; G01N 33/96; G01N 2440/12; G01N 2496/00

(57) ABSTRACT

The present invention relates to methods and uses of a biologically derived nucleosome preparation which contains a defined amount or concentration of nucleosomes expressed in absolute units of mass or concentration as a calibrant in a comparative analytical procedure.

### FIELD OF THE INVENTION

The present invention relates to the calibration of assays for cell free nucleosomes and related chromatin fragments.

### BACKGROUND OF THE INVENTION

Immunoassay methods for the estimation of nucleosomes are well known in the art, including the Cell Death ELISA (Enzyme Linked ImmunoSorbent Assay) marketed commercially by Roche Life Sciences (Holdenrieder et al, 2001) as well as assays developed by academic workers (Salgame et al, 1997; van Nieuwenhuijze et al, 2003).

Immunoassays are comparative analytical methods. In brief this means that the method does not directly establish the mass or concentration of an analyte present in a sample but provides a comparison to another sample or calibrant of known mass or concentration. As an analogy, a weighing balance is also a comparative analytical method in which the mass of a test item, on one arm of the balance, is compared to a calibrator weight on the other arm. The balance cannot directly determine the mass of the test item, but it can determine whether the mass is the same as the mass of the calibrator weight that it is compared to (for example a 1 kg weight). The determined mass of the test item will only be correct if the mass of the calibrator weight used is correct.

Immunoassays typically function by measuring the degree of antibody binding to an analyte and using this as a measure of the amount of analyte present. The degree of antibody binding is typically determined by labelling either the antibody or the antigen with a detectable tracer including for example, without limitation, a radioactive, colorimetric, chemiluminescent or fluorescent moiety which will give an assay output signal in radioactive counts, optical density (OD), relative light units (RLU) or fluorescence intensity respectively.

However, the immunoassay output signal is not a direct measurement of the mass or concentration of the analyte present in a test sample. In order to transform the assay output signal into a measure of concentration, for example in g/L or mol/L units, the signal is compared to that of a calibrant, or a series of calibrants, of known analyte concentration. The concentration of any test sample can then be determined by comparing the assay output signal observed for the test sample, to that observed for a calibrant that produces the same assay output signal in the immunoassay. In most cases a calibration curve, also often called a standard curve, is produced and the concentration of the sample is interpolated from the calibration curve. Thus, in order to provide a quantitative result, an immunoassay requires a calibrant to be used as a comparator and quantitative immunoassay kits usually include such a calibrant or a series of calibrants.

Recombinant proteins are commonly used as a calibrant material in immunoassays. This is because they can be synthesised as pure single chemical moieties and can therefore be assigned an absolute analyte value in mass or concentration terms. Prior to recombinant protein technology, immunoassays used biologically derived materials as calibrants which were assigned arbitrarily in “units”, typically defined in terms of biological activity rather than mass. For example, insulin was administered to patients in “units” rather than in grams. Similarly, proteins were, and sometimes are, measured by immunoassays in terms of “units”, usually International Units (IU), set by an authority (Henry et al, 2021). Immunoassay measurements world-wide were expressed in IU and were therefore comparative measurements only. The absolute concentration or mass of the analyte in any sample remained unknown because the concentration or mass of the IU was unknown.

Nucleosomes are not a single protein moiety that can be produced as a recombinant protein. Nucleosomes are complex nucleoprotein structures that are made up of at least 8 proteins and a length of DNA. Recombinant nucleosomes are difficult to synthesise and biologically derived calibrants are therefore normally used in nucleosome assays. However, these assays do not produce results expressed in absolute concentration or mass units but in terms of assay output signal (for example OD) or other arbitrary units (AU). See, for example, Holdenrieder et al, 2001.

Biologically derived chromatin fragments include mononucleosomes, oligonucleosomes, and larger chromatin fragments. We have previously described immunoassays for cell free nucleosomes containing particular epigenetic signals including particular histone post-translational modifications (PTMs), histone variants, DNA modifications and particular nucleosome-protein adducts, see WO 2005/019826, WO 2013/030577, WO 2013/030579 and WO 2013/084002.

Recombinant nucleosomes may be synthesised by methods known in the art. Typically, this involves the production of recombinant histones and chemically assembling individual histones into histone octamers which can be bound to suitable lengths of DNA to form recombinant nucleosomes (Dyer et al, 2004). Typically, recombinant nucleosomes are pure single molecular complexes comprising a single histone isoform combination. Recombinant nucleosomes that additionally contain a single post-translational histone modification have been described (Munari et al, 2012).

Biologically derived chromatin fragments include mononucleosomes, oligonucleosomes, Neutrophil Extracellular Trap (NET) material as well as non-histone protein-DNA complexes. Biologically derived chromatin fragments or nucleosomes are not synthesised but obtained from naturally occurring sources. Some commonly used sources of biologically derived chromatin fragments for use as immunoassay calibration materials include chicken erythrocyte nucleosomes, cell culture derived nucleosomes and human blood.

Recombinant nucleosome preparations consist of a single homogeneous nucleosome species comprising a combination of 4 pairs of histone molecules, with an identical histone isoform and histone post-translational modification (PTM) composition, bound to an oligonucleotide of defined base pair sequence and length (for example 147 base pairs). A typical recombinant nucleosome contains no histone PTMs if used as a calibrant in immunoassays for unmodified nucleosomes or a single PTM if used as a calibrant for nucleosome immunoassays containing that PTM. They typically contain no further chromatin proteins. In summary, recombinant nucleosomes are in principle ideal calibrant materials for use in comparative assays for nucleosomes containing a particular epigenetic structure or feature such as a particular histone PTM or isoform because they are a known single moiety containing the epigenetic structure of interest. However, stable pure recombinant nucleosome calibrants are difficult to manufacture reliably and reproducibly on a commercial scale.

In contrast, biologically derived nucleosome preparations are not homogeneous but comprise an unknown mixture of an unknown large number of different combinations of histone modifications and histone isoforms. There are thousands of potential combinations of histone modifications and histone isoforms that may occur in a mixture. In addition, the nucleosomes will contain different DNA fragments of different size and sequence and may or may not also be bound to other chromatin proteins (for example transcription factors). Moreover, the combinations of nucleosome structures present in any two biological preparations will differ. As biologically derived nucleosome preparations are simple and low cost to produce, they are used as relative calibrants in the great majority of nucleosome immunoassays whose results are therefore expressed in signal output or arbitrary units. However, they are not used as absolute calibrant materials for nucleosome immunoassays containing a particular epigenetic structure such as a particular histone modification or isoform defined in mass or concentration units.

We now describe an effective method for the absolute calibration of nucleosome immunoassays in units of mass or concentration using biologically derived materials. In particular, we describe immunoassays for nucleosomes containing particular histone PTM structures using biologically derived calibrants assigned or defined in absolute mass or concentration units.

### SUMMARY OF THE INVENTION

According to a first aspect of the invention, there is provided the use of a biologically derived nucleosome preparation which contains a defined amount or concentration of nucleosomes expressed in absolute units of mass or concentration as a calibrant in a comparative analytical procedure.

According to a further aspect of the invention there is provided an immunoassay method for the measurement of nucleosomes in a test sample, wherein said immunoassay is calibrated using a biologically derived nucleosome preparation assigned, or defined by, values expressed in absolute units of mass or concentration.

According to another aspect of the invention, there is provided a kit for a comparative analytical procedure, for example an immunoassay kit, comprising the biologically derived nucleosome preparation defined herein, and optionally instructions for use of the kit in a method of diagnosing and/or monitoring disease.

### BRIEF DESCRIPTION OF FIGURES

FIG. 1. Standard curves obtained by automated H3.1-nucleosome chemiluminescence immunoassay with signal output in Relative Light Units (RLU), produced using: a) a recombinant H3.1-nucleosome standard, and b) a digested Hela cell nucleosome preparation with absolute assignment of H3.1-nucleosome values as described in Example 1. C) Comparison of values obtained for test samples in immunoassays using the two standard sets. The results using the two standard sets all lie on the line of unity showing that the biologically derived standard material produced results equivalent to results obtained with recombinant nucleosome calibrant materials.

FIG. 2. Standard curves for an H3K27Me3-nucleosome automated immunoassay with signal output in RLU, produced using: a) a recombinant H3K27Me3-nucleosome standard, and b) the same digested Hela cell nucleosome preparation shown in FIG. 1 but with absolute assignment of H3K27Me3-nucleosome values as described in Example 2. C) Comparison of values obtained for test samples in immunoassays using the two standard sets are shown. The results using the two standard sets all lie on the line of unity showing that the biologically derived standard produces results equivalent to results obtained with recombinant nucleosome calibrant materials.

FIG. 3. Western blot results for the analysis of a chicken erythrocyte nucleosome preparation (produced as described in Example 3) for histone H3 and histone modification H3K27Me3. The chicken erythrocyte nucleosome preparation was analysed at dilutions 1:500, 1:1000, 1:2000 and 1:4000. The results show that the preparation contained histone H3, including histone H3K27Me3, and that the intensity of the Western blot signal is proportional to the amount of material present.

FIG. 4. An electropherogram of DNA extracted from a chicken erythrocyte nucleosome preparation (produced as described in Example 3). The electropherogram confirms that DNA was present in the chicken erythrocyte nucleosome preparation and that the DNA was comprised predominantly of fragments of 130-180 base pairs in length, corresponding to the size range expected of DNA fragments associated with mononucleosomes.

FIG. 5. Chicken erythrocyte derived absolute concentration calibration curves for (a) H3.1-nucleosome concentration and (b) H3K27Me3-nucleosome concentration, obtained by automated chemiluminescence immunoassay with Relative Light Units (RLU) as output.

### DETAILED DESCRIPTION

According to a first aspect of the invention, there is provided the use of a biologically derived nucleosome preparation which contains a defined amount or concentration of nucleosomes expressed in absolute units of mass or concentration as a calibrant in a comparative analytical procedure.

The nucleosome is the basic unit of chromatin structure and consists of a protein complex of eight highly conserved core histones (comprising of a pair of each of the histones H2A, H2B, H3 and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, Methods Mol. Biol. (2007) 361: 25-62).

Recombinant nucleosomes are chemically synthesised nucleoproteins. Methods for the preparation of recombinant nucleosomes are known in the art and typically, involve the production of the individual recombinant core histones, assembling the individual histones into histone octamers and binding the octamers to suitable lengths of DNA to form recombinant nucleosomes (Dyer et al, 2004). Typically, recombinant nucleosomes are pure single molecular complexes comprising a single histone isoform combination and are also uniform in terms of their post-translational histone modification composition.

For the purposes of the present invention, a biologically derived nucleosome is a nucleosome that is not synthesised by chemically assembling recombinant histone and DNA components, but is a natural, cell derived nucleosome produced by a living cell. Therefore, the biologically derived nucleosome used as a calibrant, as described herein, is not a recombinant nucleosome. The terms “biologically derived nucleosome”, “cell derived nucleosome” and “natural nucleosome” are used interchangeably herein.

Biologically derived, or cell derived nucleosomes may be used in a raw form, but in preferred embodiments of the invention the chromatin originating from cells is isolated, purified and fragmented (for example by sonication or nuclease digestion) to produce small chromatin fragments, including mononucleosomes and dinucleosomes, from larger cell derived chromatin fragments. In a further embodiment the biologically derived nucleosomes are cell free nucleosomes present in a body fluid, for example without limitation, lymph, cerebrospinal fluid, bronchoalveolar lavage fluid, blood, plasma or serum, wherein the cell free nucleosomes originate from (and were produced by) cells in the body. In another embodiment the biologically derived nucleosomes are cell free nucleosomes present in a cell culture supernatant, which cell free nucleosomes originate from (and were produced by) cells in culture. In one embodiment, the biologically derived nucleosomes are cell free nucleosomes present in extracellular trap (ET) material. Neutrophil extracellular trap (NET) or other ET material may be harvested from a number of sources including ex vivo culture of white blood cells stimulated to NETosis, or from body fluids such as blood, serum, plasma or other body fluids.

References to “nucleosome” may refer to “cell free nucleosome” when detected in body fluid samples. It will be appreciated that the term cell free nucleosome throughout this document is intended to include any cell free chromatin fragment that includes one or more nucleosomes. In addition, a cell free nucleosome may be a mononucleosome (analogous to a single “bead”), an oligonucleosome (analogous to a string of “beads”), part of a larger chromatin fragment or part of a NET or NET metabolite. Often the cell free nucleosomes present in a body fluid sample will be a mixture of some or all of these types.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone isoform (also referred to herein as a histone variant), a protein bound to the nucleosome (i.e. a nucleosome-protein adduct), a DNA fragment associated with the nucleosome and/or a modified nucleotide associated with the nucleosome. For example, the component thereof may be histone (isoform) H3.1, histone H1 or DNA.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. In one embodiment, the measuring of nucleosomes per se comprises detecting a core histone protein, such as detecting histone H4. As described herein, histone proteins form structural units known as nucleosomes which are used to package DNA in eukaryotic cells.

Normal cell turnover in adult humans involves the creation by cell division of a huge number of cells daily and the death of a similar number, mainly by apoptosis. During the process of apoptosis chromatin is broken down into mononucleosomes and oligonucleosomes, some of which may be released into the circulation. Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenrieder and Stieber, 2009).

Nucleosome ELISA methods were developed primarily for use in cell culture, usually as a method to detect apoptosis (Salgame et al, 1997, Holdenrieder et al, 2001, van Nieuwenhuijze et al, 2003), but are also used for the measurement of circulating cell free nucleosomes in serum and plasma (Holdenrieder et al, 2001). These assays are typical methods. The immunoassay methods of Salgame et al and van Nieuwenhuijze et al employed no calibrant and assay results were expressed in terms of assay output which was OD. Holdenrieder et al used a commercially available nucleosome immunoassay kit (Cell Death Detection plus-ELISA kit produced by Roche Diagnostics). However, the kit included no calibrant. In order to enable the relative quantification of nucleosomes and to improve intra-assay and inter-assay comparability, Holdenrieder et al produced a relative calibration material by mixing equal volumes of whole blood samples taken from 3 healthy donors and incubating the mixed blood for 3 days at 37° C., after which time the supernatant, which contained high concentrations of nucleosomes, was used as a calibrant. As the actual level of nucleosomes in this calibrant was unknown, the calibrant and the measured samples were defined in terms of arbitrary units (AU). For ELISA, the material was diluted with buffer in the range 1:24 to 1:96 to prepare a standard curve. Buffer was used as a zero standard.

A common application for immunoassays for cell free nucleosomes containing particular epigenetic structures or features, including histone PTMs or histone variants is the measurement of circulating cell free nucleosomes. Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in circulating nucleosome levels will be associated with elevations in circulating nucleosome subsets. Assays for these types of chromatin fragments are known in the art, including for example, those described in WO 2005/019826, WO 2013/030579, WO 2013/030578 and WO 2013/084002. The results of these immunoassay methods were not expressed in terms of mass or concentration but in terms of assay signal output (OD).

In order to overcome these shortcomings, we have assigned absolute values to biologically derived nucleosome preparations (also referred to herein as “material”). We assigned an absolute value in ng/ml (nanograms per millilitre) for the concentration of nucleosomes containing the histone variant H3.1 (H3.1-nucleosomes), to a nucleosome preparation produced by digestion of cellular chromatin isolated from Hela cells in cell culture. We did this by reference to a known mass of a recombinant H3.1-nucleosome in an immunoassay to define the level in the biological material. We similarly assigned an absolute value in ng/ml for the concentration of nucleosomes containing histone H3 modified by tri-methylation of the lysine residue at position 27 (H3K27Me3-nucleosomes) to the same Hela cell derived nucleosome preparation.

We then showed that use of the same (single) Hela cell calibrant in assays for both H3.1-nucleosomes and H3K27Me3-nucleosomes produced correct results for absolute nucleosome concentration in both assays. This demonstrates that biologically derived calibrant materials may be used for comparative analytical methods, including immunoassay, to establish the absolute mass or concentration of nucleosomes or nucleosomes containing a particular epigenetic signal. The advantages of this method include cost, scale and the facility to use a single calibration material for multiple analytical methods for different types of nucleosomes containing different epigenetic structures or combinations of epigenetic structures. In particular, the methods and uses described herein provide a single calibration material for methods involving different types of nucleosomes, such as those containing different epigenetic features, or nucleosomes containing multiple combinations of epigenetic features. Single biologically derived calibration materials used for multiple immunoassay methods for different related analytes with a different assigned value for each analyte are novel.

It will be understood that other methods may be used to establish the absolute concentration of a certain type of nucleosome in a biologically derived nucleosome calibrant containing a large number of different nucleosome types. In particular, mass spectrometry has been reported for the measurement of levels of nucleosomes and for the measurement of nucleosomes containing particular histone modifications or other epigenetic signals in biologically derived materials (see for example Janssen et al; 2019, Bonnet et al; 2019, Volker-Albert et al; 2018, Luense et al; 2016). Any such method for establishing, defining or assigning the absolute concentration of a calibrant may be used for the present invention, including mass spectrometry.

According to one aspect of the invention, there is provided a biologically derived nucleosome material which contains a defined amount, quantity or concentration of nucleosomes expressed in absolute units of mass or concentration for use as a calibrant in a comparative analytical procedure.

The value assigned to a calibrant of the invention may be defined in any absolute terms or S.I. units including mass terms (e.g. kilogram (kg) or gram (g)) or in mass per volume terms (e.g. in gram per litre terms) or in molar terms (e.g. moles per litre). In the Examples provided herein, we have used nanograms per millilitre (ng/ml) as absolute units.

Calibrants of the invention are useful in any comparative analytical method involving assessing the level of a nucleosome in a test sample by comparison to the known defined or assigned level present in a standard or calibrant sample. This typically involves producing a chemical or physical response from the nucleosome under investigation in the test sample and comparing the response to that produced by the standard or calibrant sample. In one embodiment, the comparative analytical procedure is for the measurement of the level of a histone post translational modification in a sample.

The test samples to be measured may be any sample comprising nucleosomes. In a preferred embodiment the test sample to be assayed by a comparative analytical method employing a calibrant or method of the invention is a human or animal body fluid sample including for example a blood, serum, plasma, cerebrospinal fluid, urine, faeces, sputum or saliva sample. Blood, serum or plasma samples are of particular interest. Therefore, in one embodiment, the biologically derived nucleosome preparation is used as a calibrant in a comparative analytical procedure which measures the level of histone post translational modifications in a blood, serum or plasma sample. Test samples may be prepared, for example where appropriate diluted or concentrated, and stored in the usual manner.

Immunoassay is a comparative analytical procedure in which the nucleosomes present in an unknown test sample may be measured using a binding agent that binds specifically to nucleosomes and the degree of binding is compared to the binding that occurs in a known calibrant or standard sample. Therefore, according to a further aspect of the invention there is provided an immunoassay method for the measurement of nucleosomes in a test sample, wherein said immunoassay is calibrated using a biologically derived nucleosome preparation assigned, or defined by, values expressed in absolute units of mass or concentration.

In one embodiment, the specific binding agent is a chromatin protein. In preferred embodiments, the specific binding agent is an antibody. In one embodiment, the immunoassay employs a single binding agent of nucleosomes. In another embodiment, the immunoassay is a 2-site immunometric (or sandwich) assay employing two binding agents, such as antibodies, directed to bind to epitopes present in a nucleosome. The antibodies or other binding agents may be directed to bind to any epitope present in a nucleosome including without limitation binding to a histone, nucleosome core or DNA epitope. In some embodiments in which nucleosome adducts are measured, one or more antibodies may be directed to bind to a protein adducted to a nucleosome.

The biologically derived cell free nucleosome material for use as a calibrant may be produced from any biological material comprising chromatin. Common sources include chicken erythrocyte nucleosomes, circulating cell nucleosomes in blood, NETs and cell culture derived nucleosomes. Methods for the preparation of cell free nucleosomes from chicken erythrocytes and cultured cells are well known in the art. Chicken erythrocyte derived and cell culture derived nucleosomes are also available commercially, for example from Tebu-bio. Another potential source of nucleosomes includes heterophils, which are a type of granulocyte found in most avian species (e.g. chickens). Similar to neutrophils, heterophils can form heterophil extracellular traps (HETs). HETs released from chicken heterophils are structurally similar to NETs found in mammalian and fish neutrophils.

Many or most types of cells, particularly vertebrate cells, may be used as a source of chromatin material for the preparation of a biologically derived nucleosome calibrant. This is because most cells contain chromatin, and histone and nucleosome structures are highly conserved across species. This means that nucleosomes derived from any convenient animal source may be used as a nucleosome calibrant material for the absolute quantitation of nucleosomes in body fluid samples obtained from individuals of that species, or other species. For example, nucleosomes derived from any convenient animal source may be used as calibrant material for the absolute quantitation of nucleosomes in human body fluid samples.

In one embodiment the nucleosomes may be provided diluted into the body fluid to be analysed. For example, animal derived nucleosomes may be provided in a matrix of human plasma.

In preferred embodiments, the biologically derived nucleosome material is produced from cells in cell culture (i.e. cultured cells). Methods for deriving cell free nucleosome material from cells in culture are well known (see for example Sadeh et al, 2016). In a typical method the chromatin material in the nucleus is isolated from the cells. This can be done using a nuclear isolation buffer or a commercially available nuclear isolation kit (for example the Merck, Nuclei PURE Prep kit). The chromatin material is then fragmented, typically by sonication to physically disrupt the chromatin, or by digestion using a nuclease (usually a micrococcal nuclease [MNase]). The chromatin fragments include mononucleosomes and/or oligonucleosomes. Optionally the chromatin may be cross-linked prior to preparation of fragmented chromatin by treating the cells with formaldehyde prior to isolation of the nuclear material. Cross-linking stabilises the fragmented chromatin and thus provides a more stable calibrant material. Alternatively, pre-prepared cross-linked fragmented chromatin materials produced from a variety of different cell types are available commercially including from Hela cervical cancer cells, HepG2 liver cancer cells, K562 leukaemia cells and 3T3 embryonic fibroblast cells. Any tissue or cultured cells may be used as a source of fragmented chromatin. Herein, we have used a chromatin fragment preparation produced from Hela cells.

As a biologically derived nucleosome preparation contains a multiplicity of nucleosome types that comprise a wide variety of epigenetic features and combinations thereof, it will be understood that nucleosomes containing any particular epigenetic feature or features will form a subgroup of nucleosomes within the overall nucleosome composition of the biologically derived preparation. A value in absolute units may be assigned to the level of any such subgroup of nucleosomes to produce a calibrant of the invention for the measurement of said subgroup of nucleosomes.

Therefore, according to a further aspect of the invention there is provided the use of a biologically derived nucleosome preparation which contains a defined amount, quantity or concentration of nucleosomes comprising one or more specified epigenetic features or signals expressed in absolute units of mass or concentration as a calibrant in a comparative analytical procedure. In some embodiments, the biologically derived nucleosome preparation contains a defined amount, quantity or concentration of nucleosomes comprising one or more specified epigenetic features or signals expressed in absolute units of mass or concentration.

As a biologically derived nucleosome preparation contains a multiplicity of nucleosome types that comprise a wide variety of epigenetic features and combinations thereof, it will also be understood that a single nucleosome preparation may be used as a calibrant for a multiplicity of different assays for the comparative measurement of a multiplicity of different nucleosome types. For this purpose, the same nucleosome preparation may be assigned calibration values for multiple nucleosome types as described herein. For example, the nucleosome preparation may be assigned a first calibration value for a first nucleosome type and second calibration value for a second nucleosome type. The number of different assays for which a single biologically derived nucleosome preparation may be used as a calibrant is in practice limited only by the number assignments made. In this regard, biological nucleosome preparations of the invention may be considered “universal” nucleosome calibrants. For example, a single nucleosome preparation may be used as a “universal” nucleosome calibrant in a multiplex assay for a multiplicity of different nucleosome types. Similarly, a single calibrant preparation may be used as a key component of multiple different immunoassay kits for different nucleosome types.

Therefore, according to another aspect of the invention there is provided a biologically derived nucleosome preparation which contains defined or assigned amounts or concentrations, expressed in absolute units of mass or concentration, of a multiplicity of nucleosome types, each comprising one or more specified epigenetic features or signals. In some embodiments, the biologically derived nucleosome preparation contains a defined amount or concentration of a type of nucleosomes comprising a specified epigenetic feature. In a further embodiment, the biologically derived nucleosome preparation contains defined amounts or concentrations of a multiplicity of nucleosome types, each comprising one or more specified epigenetic feature. Thus, in one embodiment, the biologically derived nucleosome preparation comprises defined amounts or concentrations of one or more specified epigenetic features.

In one embodiment, the multiplicity of nucleosome types with assigned absolute units, includes a value or level for nucleosomes (“all” nucleosomes, “total” nucleosomes or nucleosomes per se). This may be useful of itself and may also be used for normalisation of levels of other nucleosome types. The level of cell free nucleosomes containing a particular epigenetic feature may be normalised against the level of nucleosomes per se to express the level as a proportion of nucleosomes that contain the feature. For example, the level of nucleosomes containing the histone modification H3K4Me3 may be expressed as a percentage of nucleosomes that contain the modification. Thus, in a further embodiment, at least one of the multiplicity of nucleosome types is nucleosomes per se.

It will be understood that more than one epigenetic feature of cell free nucleosomes may be detected by immunoassay or other comparative analytical methods. Multiple biomarkers are often used in medicine, for example to determine the disease status of a subject. One advantage of a biologically derived calibrant is that it comprises a mixture of a large number of different nucleosome types so a single biologically derived calibrant may be used as a combined calibrant for a large number of different nucleosome assays either separately or in a multiplex format. Therefore, in one embodiment, the biologically derived nucleosome preparation contains a combination of multiple different nucleosomes, and optionally other chromatin fragments, comprising multiple epigenetic features as a combined calibrant. In one embodiment the calibrant is a so-called universal, or near-universal, chromatin fragment calibrant that is suitable for use in all or most nucleosome or chromatin fragment assays. The epigenetic features useful in a calibrant may be of the same type (e.g. multiple PTM types, multiple histone isoforms, multiple nucleotides or multiple protein adducts) or different types (e.g. a PTM in combination with a histone isoform).

The structure of a nucleosome may vary by the inclusion of alternative histone isoforms or variants which are different gene or splice products and have different amino acid sequences. In one embodiment, the epigenetic feature of the nucleosome, such as of one or more of the multiplicity of nucleosome types, comprises a histone variant or isoform. Many histone isoforms are known in the art. Histone isoforms can be classed into a number of families which are subdivided into individual types. The sequences of a large number of histone isoforms are known and publicly available for example in the National Human Genome Research Institute NHGRI Histone Database (Marino-Ramirez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, isoforms of histone H2 include H2A1, H2A2, mH2A1, mH2A2, H2AX and H2AZ. In another example, histone isoforms of H3 include H3.1, H3.2 and H3t. In one embodiment, the histone isoform is H3.1.

The structure of nucleosomes can vary by post translational modification (PTM) of histone proteins. PTM of histone proteins typically occurs on the tails of the core histones and common modifications include acetylation, methylation or ubiquitination of lysine residues as well as citrullination or methylation of arginine residues and phosphorylation of serine residues and many others. It will be understood that a histone PTM may occur on different isoforms (variants) of the histone. For example, the lysine residues that occur on the tail of histone H3 isoforms H3.1, H3.2 and H3.3 may be modified by acetylation or methylation. Many histone modifications are known in the art and the number is increasing as new modifications are identified (Zhao and Garcia; 2015). Therefore, in one embodiment, the epigenetic feature of the cell free nucleosome may be a histone post translational modification (PTM). The histone PTM may be present on a core nucleosome histone (e.g. H2A, H2B, H3 or H4), or a linker histone (e.g. H1 or H5). Examples of PTMs are described in WO 2005/019826 and WO 2017/068359.

In one embodiment, the biologically derived nucleosome preparation may be assigned amount, concentration or mass units for nucleosomes containing particular histone PTMs including acetylation, methylation (which may be mono-, di- or tri-methylation), phosphorylation, ribosylation, citrullination, ubiquitination, hydroxylation, glycosylation, nitrosylation, glutamination and/or isomerisation. This may then be used to determine the level of histone post translational modification in a sample (e.g. a blood, serum or plasma sample from a patient). In one embodiment, the histone PTM is selected from citrullination or ribosylation. In a further embodiment, the histone PTM is citrullinated H3 (H3cit) or citrullinated H4 (H4cit). In a yet further embodiment, the histone PTM is citrullination of the arginine residue at amino acid position 8 of histone H3 (H3R8cit).

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes and without limitation, anti-pan-acetylation antibodies (e.g. a Pan-acetyl H4 antibody [H4panAc]), anti-citrullination antibodies or anti-ubiquitin antibodies.

In one embodiment, the epigenetic feature is a DNA modification, thus the epigenetic feature of the nucleosome calibrant with a defined mass or concentration comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues, or 5-hydroxymethylcytosine residues or other nucleotides or modified nucleotides, than other nucleosomes. In one embodiment, the epigenetic feature is a DNA modification selected from 5-methylcytosine or 5-hydroxymethylcytosine. Thus, in some embodiments, the defined calibrated DNA modification is 5-methylcytosine or 5-hydroxymethylcytosine.

A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art. Therefore, in one embodiment, the epigenetic feature comprises one or more protein-nucleosome adducts or complexes. In a further embodiment, the epigenetic feature of the calibrant with a defined mass or concentration is one or more protein-nucleosome adducts or complexes.

Defined mass or concentration values or units may be assigned to a biologically derived nucleosome calibrant for any single nucleosome moiety, type or subgroup, or any combination of a multiplicity of nucleosome moieties, types or subgroups as described herein. The terms “type” and “moiety” as used in the context of nucleosomes herein will be understood to refer to the identity of the nucleosome or the way in which it is identified, for example a first ‘type’ of nucleosome may be identified by detecting nucleosomes per se, while a second ‘type’ of nucleosome may be identified by detecting a post translational modification and/or a particular histone isoform, and a yet further third ‘type’ of nucleosome may be identified by detecting an epigenetic feature such as a DNA modification.

Therefore, according to a further aspect of the invention there is provided a method for assigning an absolute unit of mass or concentration to a biologically derived nucleosome preparation, said method comprising:
(i) obtaining a cell nuclear extract from a biological source, such as cells grown in cell culture;
(ii) digesting the cell nuclear extract using nuclease to obtain a biologically derived mono-nucleosome preparation;
(iii) diluting the biologically derived mono-nucleosome preparation at various dilution factors in lipid depleted plasma; and
(iv) assigning an absolute unit of mass or concentration to each of the dilution factors of biologically derived mono-nucleosome preparation by measuring the level of nucleosomes in an immunoassay and comparing the luminescence measured for each dilution factor with the luminescence measured for a set of standard solutions consisting of a defined amount or concentration of recombinant nucleosomes, said defined amount expressed in absolute units of mass or concentration.

One disadvantage of recombinant nucleosome moieties for use as calibrant materials is that they are not representative of the biological nucleosomes to be measured in a biological sample. For example, histone H3 includes lysine residues at positions K4, K9, K14, K18, K23, K27, K36, K56 and K79. Each of these lysine residues may be unmodified, acetylated, mono-methylated, di-methylated, tri-methylated, ubiquitinylated, sumoylated or modified in other ways. Many other histone modifications also occur. Moreover, these modifications may occur on different histone isoforms. Biological nucleosomes in a biological sample comprise a complex mix of many different combinations of many histone modifications on different histone isoforms. In contrast, an immunoassay for total nucleosomes (or nucleosomes per se) may employ a pure single unmodified recombinant nucleosome calibrant consisting of a single set of unmodified histone isoforms of H2A, H2B, H3 and H4. Therefore, the recombinant nucleosome used as calibrant is not representative of nucleosomes in a biological sample. Indeed, it is unlikely that a single such nucleosome exists in the sample to be assayed.

Moreover, because the biological sample includes a vast and complex mix of an enormous number of different nucleosome structures, it is unlikely that any single nucleosome structure can be representative of the nucleosomes present in a biological sample.

Similarly, any assay for a nucleosome containing a particular epigenetic structure, for example a histone isoform or a histone modification such as H3K9Ac, may employ a single pure recombinant nucleosome containing (only) the histone modification H3K9Ac but which is otherwise devoid of histone modifications. Again, this recombinant nucleosome is not representative of biological nucleosomes and it is unlikely that a single such nucleosome exists in a biological sample for assay.

We have found that recombinant nucleosomes have different analytical recoveries in different nucleosome assays employing recombinant nucleosome calibrants. Moreover, we found that the different recoveries are often not constant but dose dependent. This may be illustrated by reference to a hypothetical assay for total nucleosomes employing an antibody directed to bind to an epitope, for example, near to amino acid residues 9-14 on histone H3 together with an unmodified recombinant nucleosome calibrant. Different recombinant nucleosomes, each containing any single histone modification selected from H3K9Ac, H3K9Me3, H3K14Ac, H3K14Me3 or any other possible modification at these 2 positions, may all give different signals to each other, as well as to the unmodified recombinant nucleosome calibrant, and therefore different analytical recoveries, in such an assay. A similar effect will occur for the cross-reactivity of different recombinant nucleosomes in assays for nucleosomes containing a particular epigenetic structure such as a particular histone modification or histone isoform.

Similarly, the signal produced by a biological nucleosome in an immunoassay for nucleosomes containing H3K9Ac, for example, may be affected by the many possible modification states of amino acids at nearby positions. Considering, for simplicity, only the adjacent amino acid residues at positions 8 and 10, biological nucleosomes containing any combination of any of the modifications citrullinated-R8, methylated-R8 or phosphorylated-S10 (in addition to H3K9Ac) may react differently to an antibody directed to bind to H3K9Ac. This effect is not limited to any example pair of modifications but extends to many potential modification sites present in a biological nucleosome, each of which may be modified in multiple ways. An advantage of a biologically sourced calibrant material is that the calibrant is more representative of the complex mix of nucleosome structures present in a biological sample and will therefore illicit an assay response that is more representative of that elicited by nucleosomes in the samples to be assayed. Moreover, a single calibrant may be used for multiple different nucleosome assays and therefore provide a consistent recovery that relates to the mix of biological structures.

Assays for nucleosomes have been investigated for use as biomarkers for many diseases including cancer, inflammatory, autoimmune, infectious and NETosis related conditions. Therefore, in one aspect of the invention there is provided the use of a biologically derived nucleosome calibrant preparation defined in absolute units of mass or concentration in a clinical diagnostic test for a human or animal subject.

Histone modifications are not equally distributed in chromatin and some modifications will therefore occur more frequently than others in a biological sample. Where a calibrant is being produced for a nucleosome moiety containing an uncommon histone modification, samples of biologically derived nucleosomes may contain a low, or a very low, proportion of the desired nucleosomes. Thus, the preparation of biologically derived calibration materials for nucleosomes containing some histone modifications, may require enrichment for those nucleosomes. Such enrichment may be effected by a number of means including; (i) isolation or purification of a nucleosome preparation for the desired nucleosomes; (ii) exposure of source nucleosome material to histone modifying enzymes (i.e. to produce nucleosomes with the desired modifications); (iii) modification (e.g. genetic modification or transient transfection) of cultured cells to over-express histone modifying enzymes (i.e. so that nucleosomes with the desired modifications are produced in greater quantities by the cultured cells); or (iv) treatment of cultured cells with a compound that modulates (i.e. increases or decreases) histone modifying enzyme activity or co-factors for enzymatic activity (v) any combination of these methods. These methods will be described in more detail below.

In one embodiment, a biologically derived nucleosome preparation enriched for nucleosomes containing a particular (low abundance) histone modification is prepared by isolation of, or purification for, the desired nucleosomes. For example, in a positive enrichment embodiment this is achieved by chromatin immunoprecipitation of nucleosomes containing the histone modification of interest. Methods for chromatin immunoprecipitation are well known in the art. In a typical method, an antibody that binds selectively to the histone modification of interest is immobilised on a solid phase support (for example, without limitation, agarose or sepharose or other particulate materials) and contacted with the source nucleosome material. Nucleosomes containing the histone modification of interest are isolated from other nucleosomes on the solid phase. The solid phase nucleosomes may be used in suspension as a calibrant (i.e. as the biologically derived nucleosome preparation). Alternatively, the purified nucleosomes may be removed from the solid phase for use as a liquid phase calibrant. Such an enrichment process may be applied to a nucleosome preparation derived from any natural source.

In a negative enrichment embodiment, a particular histone modification is enriched by removal or depletion of other nucleosomes from the preparation. In this embodiment an antibody that selectively binds to a nucleosome not containing the histone modification of interest is immobilised on a solid phase. The solid phase is contacted with the source nucleosome material and nucleosomes not containing the histone modification of interest are removed from the nucleosome preparation, thus enriching the preparation for nucleosomes containing the desired histone modification. For illustrative purposes only, an example of how this may be achieved is to select an antibody, or antibodies, that bind to other possible state(s) of the histone at the (same) amino acid position(s). For example, if the nucleosomes of interest were those containing histone H3 acetylated at lysine 9 (H3K9Ac), then antibodies binding to any or all of unmodified H3K9, any methylated (Me) H3K9 moiety (H3K9Me, H3K9Me2 or H3K9Me3) or any other post translationally modified (PTM) lysine moiety. Many such post translational modifications of lysine are known in the art. Such an enrichment process may be applied to a nucleosome preparation derived from any natural source.

In a further embodiment, a biologically derived nucleosome preparation enriched for nucleosomes containing a particular histone modification is prepared by enzymatic conversion of (other) nucleosomes into the nucleosomes of interest. This may be achieved using enzymes known in the art including, without limitation, histone methyl transferase (HMT), histone acetyl transferase (HAT), histone demethylase, histone deacetylase or other histone modifying enzymes. A large number of histone PTMs are known in the art and any enzyme effecting any PTM may be used for this purpose in methods of the invention. In this embodiment a histone modifying enzyme is added to a biologically derived nucleosome preparation which acts as a substrate for the enzyme. It will be understood that the histone modifying enzyme may also be applied directed to a cell culture from which the nucleosome preparation is subsequently derived. The presence of the enzyme alters the PTM of histones within the nucleosomes. Selection of the appropriate enzyme(s) used leads to conversion of nucleosome associated histone to the histone modification of interest. Using the same example (H3K9Ac) as above: a biologically derived nucleosome preparation may be enriched for nucleosomes containing this PTM using a HAT enzyme, optionally in combination with other enzymes (for example a histone demethylase enzyme). Such an enrichment process may be applied to a nucleosome preparation derived from any natural source.

In a further embodiment, cells in culture are transiently or stably genetically modified so that they over-express one or more histone modifying enzymes. The genetically modified cell line may be produced in any way known in the art, including by stably incorporating suitable gene sequences encoding histone modifying enzymes into the genome of the cell or by transfecting the cells with a suitable vector or plasmid containing such gene sequences. The advantage of this embodiment is that chromatin harvested from the cell line may be enriched for particular post-translational modifications. Many such histone modifying enzymes are known in the art and any histone modifying enzyme may be used for the methods described herein. Using the same example (H3K9Ac) as above: a cell may be engineered to produce chromatin containing elevated levels of nucleosomes containing H3K9Ac by stable or transient transfection of genetic sequences encoding a HAT enzyme, optionally in combination with genetic sequences for other enzymes (for example a histone demethylase enzyme).

In a further embodiment, a genetically modified animal is produced that over expresses one or more histone modifying enzymes. Chicken erythrocyte cells are a commonly used source of biologically derived nucleosomes. Thus, for example, a genetically modified chicken may be used to produce nucleosomes that contain high level(s) of particular histone modification(s).

In one embodiment, cells in culture are treated with a compound that modulates (i.e. increases or decreases) histone modifying enzyme activity or co-factors for enzymatic activity. Such compounds include, for example, histone methyltransferase and demethylase inhibitors.

The term “biomarker” means a distinctive biological or biologically derived indicator of a process, event, or condition. Biomarkers can be used in methods of diagnosis, e.g. clinical screening, and prognosis assessment, in monitoring the results of therapy and for identifying patients most likely to respond to a particular therapeutic treatment. Biomarkers and uses thereof are valuable for identification of new drug treatments and for discovery of new targets for drug treatment.

Comparative methods for quantifying biomarkers are known in the art. The reagents used in such methods may comprise one or more ligands, binders or binding agents, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. Thus, in one embodiment, the binding agent is an antibody. The antibody can be a monoclonal antibody or a fragment thereof. It will be understood that if an antibody fragment is used then it retains the ability to bind the biomarker so that the biomarker may be detected (in accordance with the present invention). A ligand or binder may be labelled with a detectable marker, such as a luminescent, fluorescent, enzyme or radioactive marker. Alternatively or additionally, a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

The terms “detecting” or “diagnosing” as used herein in the context of disease encompass identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, including for drug screening and drug development.

In one embodiment, the assay using a biologically derived nucleosome calibrant defined in absolute units of mass or concentration as described herein, is repeated on multiple occasions. This embodiment provides the advantage of allowing the detection results to be monitored over a time period. Such an arrangement will provide the benefit of monitoring or assessing the efficacy of treatment of a disease state. Such monitoring methods can be used to monitor onset, progression, stabilisation, amelioration, relapse and/or remission of a disease condition.

In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and at a later stage of therapy. Monitoring may comprise detecting a change in the nature or amount of the biomarker(s) in test samples taken on different occasions.

A change in the level of the biomarker in the test sample relative to the level in a previous test sample taken earlier from the same test subject may be indicative of a beneficial effect (e.g. stabilisation or improvement) of the therapy on the disorder or suspected disorder. Furthermore, once treatment has been completed, the biomarker measurement(s) may be periodically repeated in order to monitor for the recurrence of a disease.

Methods for monitoring efficacy of a therapy can be used to monitor the therapeutic effectiveness of existing therapies and new therapies in human subjects and in non-human animals. These monitoring methods can be incorporated into screens for new drug substances and combinations of substances.

Therefore, in one aspect of the invention there is provided a comparative analytical procedure kit, such as a diagnostic or monitoring kit, comprising a biologically derived nucleosome calibrant as described herein. In one embodiment, the comparative analytical procedure kit is a nucleosome immunoassay kit. Comparative analytical procedure kits, such as nucleosome immunoassay kits, will typically additionally comprise one or more ligands or binding agents for detection and/or quantification of the nucleosome biomarker, and/or a biosensor and/or an array, optionally together with instructions for use of the kit, such as instructions for use of the kit in a method of diagnosing and/or monitoring disease. Thus, in a further embodiment, the kit is a nucleosome immunoassay kit comprising one or more binding agents for detection and/or quantification of nucleosomes.

In one embodiment, the immunoassay comprising a biologically derived nucleosome calibrant defined in absolute units of mass or concentration, is a biosensor capable of detecting and/or quantifying one or more nucleosome types. As used herein, the term “biosensor” means anything capable of detecting the presence of a nucleosome. Biosensors may comprise a ligand binder or ligands, as described herein, capable of specific binding to a nucleosome. Such biosensors are useful in detecting and/or quantifying nucleosomes using calibrants of the invention.

Suitably, biosensors for detection of one or more nucleosome types combine biomolecular recognition with appropriate means to convert detection of the presence, or quantitation, of the nucleosome type in the sample into a signal. Biosensors can be adapted for “alternate site” diagnostic testing, e.g. in the ward, outpatients' department, surgery, home, field and workplace. Biosensors to detect one or more nucleosome types include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more nucleosome types.

The immunoassays described herein comprising a biologically derived nucleosome calibrant preparation defined in absolute units of mass or concentration, include any method employing one or more antibodies or other specific binders/binding agents directed to bind to the nucleosomes or nucleosome types defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels. All of said immunoassay methods are well known in the art.

Identifying, detecting and/or quantifying of nucleosomes by comparison to a biologically derived nucleosome calibrant can be performed by any method suitable to identify the presence and/or amount of a specific nucleosome type(s) in a biological sample from a subject or a purification or extract of a biological sample or a dilution thereof. In particular, quantifying may be performed by measuring the concentration of the target nucleosomes in the sample or samples.

For example, calibrants of the invention may be used in one or more method(s) selected from the group consisting of: immunoassay, immunochromatography, a 1-D gel-based analysis, a 2-D gel-based analysis, reverse phase (RP) LC, size permeation (gel filtration), ion exchange, affinity chromatography, liquid chromatography (e.g. high pressure liquid chromatography (HPLC) or low pressure liquid chromatography (LPLC)) or thin-layer chromatography.

Methods involving detection and/or quantification of nucleosomes by comparison to a biologically derived nucleosome calibrant can be performed on bench-top instruments, or can be incorporated onto disposable, diagnostic or monitoring platforms that can be used in a non-laboratory environment, e.g. in the physician's office or at the subject's home or bedside. Suitable biosensors include, without limitation, lateral flow tests and “credit” cards with optical or acoustic readers. Biosensors can be configured to allow the data collected to be electronically transmitted to the physician for interpretation and thus can form the basis for e-medicine.

The identification of nucleosome biomarkers for a disease state permits integration of diagnostic procedures and therapeutic regimes. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus, nucleosome immunoassays of the invention comprising a biologically derived nucleosome calibrant defined in absolute units of mass or concentration, can be tailored precisely to match the needs determined by the disorder and the pharmacogenomic profile of the subject, the nucleosome biomarkers selected can thus be used to titrate the optimal dose, predict a positive therapeutic response and identify those subjects at high risk of severe side effects.

The invention will now be illustrated with reference to the following non-limiting examples.

### Example 1

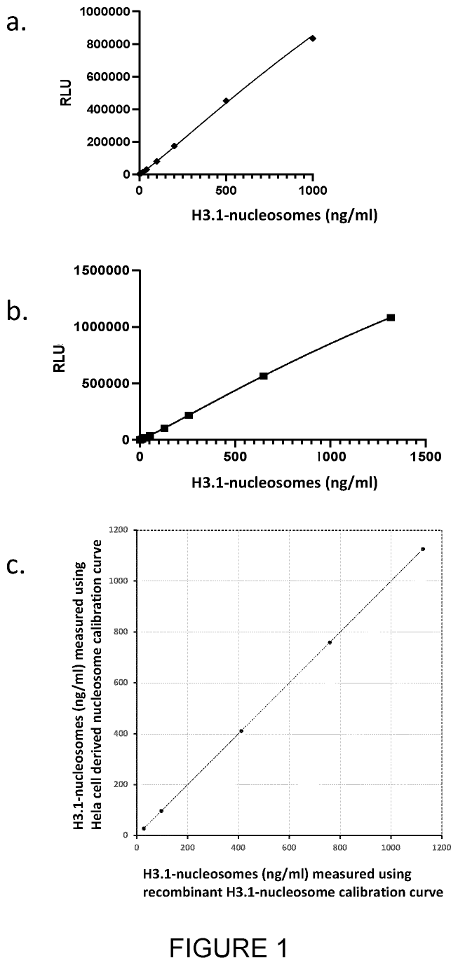

Recombinant H3.1-nucleosomes were prepared by assembly of recombinant histone proteins H2A, H2B, H3.1 and H4 together with a 147 base pair DNA oligonucleotide by methods known in the art. A 1000 ng/ml solution of recombinant H3.1-nucleosomes was prepared in lipid depleted plasma. This solution was diluted to prepare further solutions containing 500, 200, 100, 40, 20, 8, 4, 2 and zero ng/ml nucleosomes for use as a set of 10 standards (including a zero standard) to generate a recombinant H3.1-nucleosome standard curve (FIG. 1a).

Biologically derived mono-nucleosomes can be prepared by a number of methods known in the art including nuclease digestion of cell nuclear extracts from cells grown in cell culture. We diluted a commercially available Hela cell mono-nucleosome preparation in lipid depleted plasma at various levels. Hela mono-nucleosomes consist of a mixture of a very large number of different epigenetic types of nucleosomes including H3.1-nucleosomes. We measured the level of H3.1-nucleosomes present in each Hela cell nucleosome solution by means of an immunoassay using the recombinant H3.1-nucleosome set of standards described above. The concentrations measured were 1315.1, 648.8, 257.0, 129.0, 51.7, 27.0, 11.7, 6.4, 3.8 and zero ng/ml. These measured values were assigned as the H3.1-nucleosome values of the Hela cell mono-nucleosome solutions. The assigned H3.1-nucleosome values were used to generate a 10-point biologically derived H3.1-nucleosome standard curve (FIG. 1b).

The recombinant and biologically derived H3.1 nucleosome standards were then used to determine the absolute concentration of H3.1-nucleosome present in 5 test samples by immunoassay. The test sample results for the two assays correlated well with almost no bias or intercept (Table 1 and FIG. 1c).

TABLE 1
H3.1-nucleosome immunoassay results obtained using a recombinant
or biologically derived H3.1-nucleosome standard.

| | Assay result with | Assay result with | |
|---|---|---|---|
| | recombinant H3.1- | Hela cell H3.1- | |
| Sample | nucleosome standard | nucleosome standard | Bias |
| 1 | 97.0 | 97.1 | 0.0% |
| 2 | 411.8 | 411.3 | −0.1% |
| 3 | 27.8 | 27.8 | 0.2% |
| 4 | 1125.9 | 1126.2 | 0.0% |
| 5 | 759.4 | 758.9 | −0.1% |

The immunoassays were performed using an automated immunoassay system. Briefly, calibrant or sample (50 μl) was incubated with an acridinium ester labelled anti-nucleosome antibody (50 μl) and assay buffer (100 μl) for 1800 seconds at 37° C. Magnetic beads coated with an anti-histone H3.1 antibody (20 μl) were added and the mixture was incubated for a further 900 seconds. The magnetic beads were then isolated, washed 3 times and magnetic bound acridinium ester was determined by luminescence output over 7000 milliseconds in RLU.

### Example 2

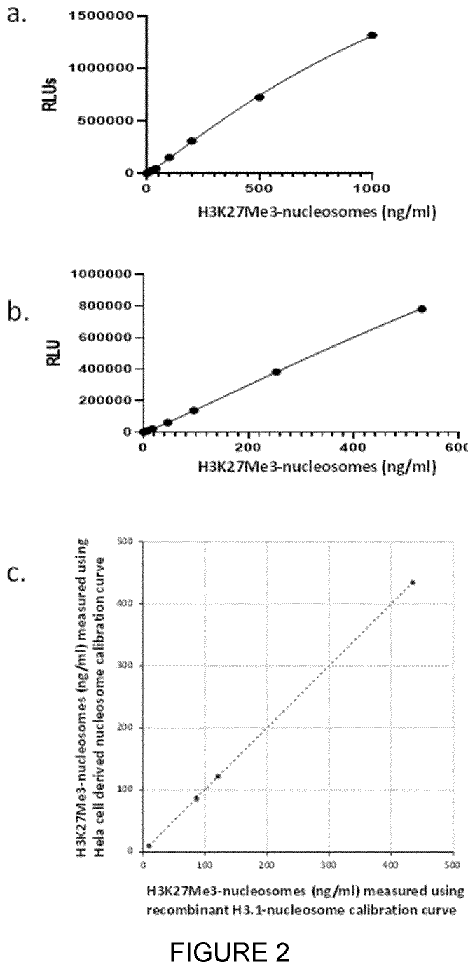

A preparation of recombinant nucleosomes containing the histone modification H3K27Me3 (tri-methylation of lysine 27 of histone H3) was assembled from recombinant histone proteins H2A, H2B, H3 and H4 together with a 147 base pair DNA oligonucleotide by methods known in the art. A 1000 ng/ml solution of recombinant H3K27Me3-nucleosomes was prepared in lipid depleted plasma. This solution was diluted to prepare further solutions for use as a set of standards to generate a recombinant H3K27Me3-nucleosome standard curve with concentrations containing 1000, 500, 200, 100, 40, 20, 8, 4, 2 and zero ng/ml (FIG. 2a).

We then measured the level of H3K27Me3-nucleosomes present in the same Hela cell nucleosome solutions described in Example 1 by means of an immunoassay using recombinant H3K27Me3-nucleosome standards. The levels measured were assigned as the H3K27Me3-nucleosome values of the Hela cell mono-nucleosome solutions. The assigned H3K27Me3-nucleosome values were used to generate a 10-point biologically derived H3K27Me3-nucleosome standard curve with concentrations containing 529.9, 252.7, 95.8, 46.0, 16.9, 8.1, 3.3, 1.8, 0.9 and zero ng/ml (FIG. 2b).

The recombinant and biologically derived H3K27Me3-nucleosome standards were used to determine the absolute concentration of H3K27Me3-nucleosomes present in 4 test samples by immunoassay. The test sample results for the two assays correlated well with almost no bias or intercept (Table 2 and FIG. 2c). The results show that the method of the invention is effective for the use of biological standards for the measurement of test samples with results expressed in absolute units of concentration or mass. The results also show that a single biologically derived standard or calibrant can be assigned values for multiple (possibly hundreds) of different types of nucleosomes containing different epigenetic structures or signals.

TABLE 2
H3K27Me3-nucleosome immunoassay results obtained
using a recombinant or biologically derived
H3K27Me3-nucleosome standard.

| | Assay result with | Assay result with | |
|---|---|---|---|
| | recombinant H3K27Me3- | Hela cell H3K27Me3- | |
| Sample | nucleosome standard | nucleosome standard | Bias |
| 1 | 10.1 | 10.0 | −0.8% |
| 2 | 121.2 | 122.0 | 0.7% |
| 3 | 86.4 | 86.9 | 0.5% |
| 4 | 434.4 | 434.4 | 0.0% |

The immunoassays were performed using an automated immunoassay system as described in Example 1 but using magnetic beads coated with an antibody directed to bind to the histone modification H3K27Me3.

### Example 3

In order to produce an animal derived nucleosome calibrant preparation we purchased chicken erythrocytes from Tebu-bio (Chicken RBC 10% washed pooled cells, catalogue number R401-0100). A predominantly mononucleosome preparation was produced from chicken erythrocytes using standard methods in the art. In brief, the cells were spun down by centrifugation at 1000 g for 10 minutes at 4° C. The supernatant buffer was removed, the cells were resuspended in buffer and spun down again by centrifugation at 1000 g for 10 minutes at 4° C. The supernatant buffer was removed and the cells were resuspended in 2-5 volumes of a lysis buffer (750 μL of 10 mM HEPES pH 7.4, 10 mM KCl, 1 mM sodium orthovanadate, 0.05% NP-40 for 50 million cells) for 20 minutes on ice to lyse the cells. The nuclei of the lysed cells were spun down by centrifugation at 12000 g for 10 minutes at 4° C. The supernatant containing the cytoplasm was removed and the nuclei were resuspended in 2-5 volumes of a low salt buffer (10 mM Tris-HCl pH 7.4; 0.2 mM MgCl₂, 1 mM sodium orthovanadate, 1% Triton) for 15 minutes on ice to lyse the nuclei. Chromatin from the lysed nuclei was isolated by centrifugation as a pellet at 12000 g for 10 minutes at 4° C. The supernatant was removed and the chromatin in the pellet was resuspended in digestion buffer (5 mM CaCl₂, 50 mM Tris-HCl pH 8, 1 mM sodium orthovanadate) containing 50units of micrococcal nuclease (MNase) for 15 minutes at 37° C. to digest the chromatin into mononucleosomes. The digestion reaction was stopped by addition of a stop solution (500 mM EDTA pH 8) and incubating 5 minutes on ice. Undigested chromatin and other solid matter was removed by centrifugation at 9000 g for 5 minutes at 4° C. The supernatant containing soluble small chromatin fragments, predominantly mononucleosomes, was retained as a biologically derived nucleosome calibrant material.

A valid biologically derived nucleosome calibrant suitable for use as calibrant for multiple assays for nucleosomes containing a variety of different histone PTMs, should contain intact mononucleosomes comprising histone protein and DNA. Ideally the component histones should retain histone PTMs present in the cells. The DNA should ideally comprise predominantly approximately 147 base pair DNA fragments corresponding to the fragment size characteristic of mononucleosomes. We therefore tested the preparation produced here for histone content, modified (PTM) histone content, DNA content and DNA fragment size.

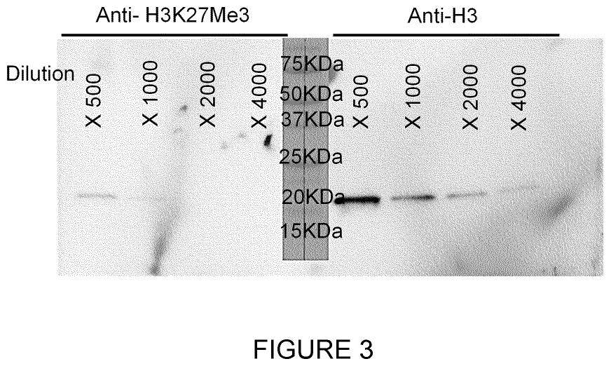

We tested the chicken erythrocyte nucleosome preparation produced here for histone content by Western blot using an anti-Histone H3 antibody, as well as an anti-H3K27Me3 antibody, by standard methods. The preparation was tested at serial dilutions at 1:500, 1:1000, 1:2000 and 1:4000. The results are shown in FIG. 3 and show that the preparation contained histone H3, including histone H3 tri-methylated at lysine 27. Moreover, the intensity of the Western blot signal decreased with dilution of the preparation.

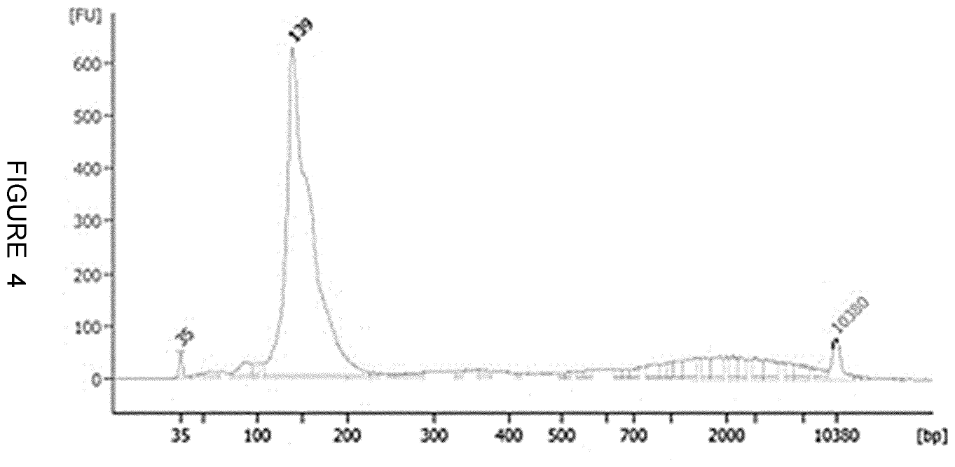

We tested the chicken erythrocyte nucleosome preparation produced here for DNA content and fragment size distribution. DNA was extracted from the preparation and analysed for its fragment size profile by electrophoresis using a Bioanalyzer (Agilent). The Bioanalyzer electropherogram produced is shown in FIG. 4 and shows that DNA was present in the chicken erythrocyte nucleosome preparation and was comprised predominantly of DNA fragments of 130-180 base pairs in length. This corresponds to the size range expected of DNA fragments associated with mononucleosomes.

We conclude that the chicken erythrocyte nucleosome preparation was composed primarily of mononucleosomes that contained DNA and modified histones.

### Example 4

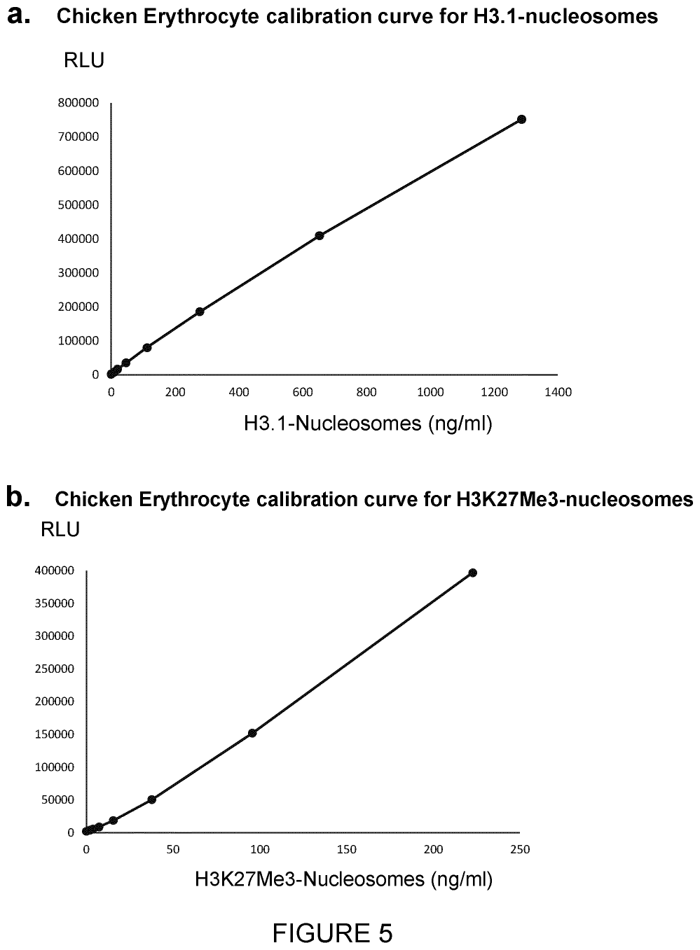

The chicken erythrocyte derived biological nucleosome calibrant material prepared in EXAMPLE 3 was assigned absolute standard concentration values for H3.1-nucleosomes and for H3K27Me3-nucleosomes by the same method used for Hela cell derived nucleosomes as described in EXAMPLES 1 and 2, respectively. The resulting chicken erythrocyte derived nucleosome standard curves in absolute units (ng/ml) are shown in FIG. 5a for H3.1-nucleosomes and FIG. 5b for H3K27Me3-nucleosomes.

A number of test samples were measured for their H3.1-nucleosome content or H3K27Me3-nucleosome content by immunoassay and the assay results are shown below in Table 3 for H3.1-nucleosome immunoassay results and in Table 4 for H3K27Me3-nucleosome immunoassay results.

TABLE 3
H3.1-nucleosome immunoassay results obtained
using a recombinant or biologically derived
chicken erythrocyte H3.1-nucleosome standard.

| | Assay result | Assay result | |
|---|---|---|---|
| | recombinant H3.1- | chicken erythrocyte | |
| Sample | nucleosome standard | H3.1-nucleosome standard | Bias |
| 1 | 324.3 | 324.3 | <0.01% |
| 2 | 51.5 | 51.5 | <0.01% |
| 3 | 83.9 | 83.9 | <0.01% |
| 4 | 70.8 | 70.8 | <0.01% |
| 5 | 49.2 | 49.2 | <0.01% |
| 6 | 44.5 | 44.5 | <0.01% |

TABLE 4
H3K27Me3-nucleosome immunoassay results obtained
using a recombinant or biologically derived chicken
erythrocyte H3K27Me3-nucleosome standard.

| | Assay result | Assay result chicken | |
|---|---|---|---|
| | recombinant H3K27Me3- | erythrocyte H3K27Me3- | |
| Sample | nucleosome standard | nucleosome standard | Bias |
| 1 | 34.9 | 34.9 | <0.01% |
| 2 | 45.3 | 45.3 | <0.01% |
| 3 | 50.7 | 50.7 | <0.01% |
| 4 | 41.2 | 41.2 | <0.01% |

The results confirm that a chicken erythrocyte derived nucleosome preparation may be used as an absolute concentration calibrant material for the absolute quantitation of nucleosomes by immunoassay and that the same material may be used as calibrant in multiple assays.

### REFERENCES

Bonnet et al, Quantification of Proteins and Histone Marks in Drosophila Embryos Reveals Stoichiometric Relationships Impacting Chromatin Regulation. doi.org/10.1016/j.devcel.2019.09.011, 2019
Dyer et al, Reconstitution of Nucleosome Core Particles from Recombinant Histones and DNA. doi.org/10.1016/S0076-6879(03)75002-2, 2003
Henry et al, Addition of Regular Insulin to Ternary Parenteral Nutrition: A Stability Study. doi.org/10.3390/pharmaceutics13040458, 2021
Herranz and Esteller, DNA methylation and histone modifications in patients with cancer: potential prognostic and therapeutic targets. Methods Mol Biol.361:25-62, 2007
Holdenrieder et al, Nucleosomes in serum of patients with benign and malignant diseases. Int. J. Cancer (Pred. Oncol.): 95, 114-120, 2001
Janssen et al, Quantitation of Single and Combinatorial Histone Modifications by Integrated Chromatography of Bottom-up Peptides and Middle-down Polypeptide Tails. doi: 10.1007/s13361-019-02303-6, 2019
Luense et al, Comprehensive analysis of histone post-translational modifications in mouse and human male germ cells. doi:10.1186/s13072-016-0072-6, 2016
Munari et al, Methylation of Lysine 9 in Histone H3 Directs Alternative Modes of Highly Dynamic Interaction of Heterochromatin Protein hHP1P with the Nucleosome. doi:10.1074/jbc.M112.390849, 2012
Holdenrieder and Stieber, Clinical use of circulating nucleosomes. Critical Reviews in Clinical Laboratory Sciences; 46(1): 1-24, 2009
Sadeh et al, Elucidating Combinatorial Chromatin States at Single-Nucleosome Resolution. doi: 10.1016/j.molcel.2016.07.023, 2016
Salgame et al, An ELISA for detection of apoptosis. Nucleic Acids Research, 25(3), 680-681, 1997
van Nieuwenhuijze et al, Time between onset of apoptosis and release of nucleosomes from apoptotic cells: putative implications for systemic lupus erythematosus. Ann Rheum Dis; 62: 10-14, 2003
Volker-Albert et al. Analysis of Histone Modifications by Mass Spectrometry. doi.org/10.1002/cpps.54, 2018
Zhao and Garcia, Comprehensive Catalog of Currently Documented Histone Modifications doi:10.1101/cshperspect.a025064, 2015

1. A method, comprising:
providing a biologically derived nucleosome preparation which contains a defined amount or concentration of nucleosomes expressed in absolute units of mass or concentration; and
using the preparation as a calibrant in a comparative analytical procedure.

2. The method of claim 1, wherein the comparative analytical procedure is for the measurement of a level of a histone post translational modification in a sample.

3. The method of claim 1, wherein the biologically derived nucleosome preparation contains a defined amount or concentration of a type of nucleosomes comprising a specified epigenetic feature.

4. The method of claim 1, wherein the biologically derived nucleosome preparation contains defined amounts or concentrations of a multiplicity of nucleosome types, each comprising one or more specified epigenetic feature.

5. The method of claim 4, wherein the biologically derived nucleosome preparation contains a combination of multiple different nucleosome types, and optionally other chromatin fragments, comprising multiple epigenetic features as a combined calibrant.

6. The method of claim 4, wherein the one or more specified epigenetic feature is selected from the group consisting of a histone post translational modification, a histone variant or isoform, a nucleotide, a modified nucleotide and a protein-nucleosome adduct.

7. The method of claim 6, wherein the histone isoform is H3.1.

8. The method of claim 6, wherein the histone post translational modification is selected from the group consisting of acetylation of lysine residues, methylation of lysine residues, ubiquitination of lysine residues, citrullination of arginine residues, methylation of arginine residues, and phosphorylation of serine residues.

9. The method of claim 8, wherein the histone post translational modification is methylation of a lysine residue.

10. The method of claim 4, wherein at least one of the multiplicity of nucleosome types is nucleosomes per se.

11. The method of claim 1, wherein the biologically derived nucleosome preparation is produced from cells in cell culture.

12. The method of claim 1, wherein the biologically derived nucleosome preparation is prepared by a method selected from the group consisting of (i) isolation or purification of a nucleosome preparation for the desired nucleosomes; (ii) exposure of source nucleosome material to histone modifying enzymes; (iii) modification of cultured cells to over-express histone modifying enzymes; and (iv) treatment of cultured cells with a compound that modulates histone modifying enzyme activity.

13. The method of claim 1, wherein the comparative analytical procedure is an immunoassay or clinical diagnostic test for a human or animal subject.

14. (canceled)

15. The method of claim 1, wherein the comparative analytical procedure comprises assessing a level of a nucleosome in a test sample.

16. The method of claim 15, wherein the test sample is a human or animal body fluid sample, such as a blood, serum, plasma, cerebrospinal fluid, urine, faeces, sputum or saliva sample.

17. An immunoassay method for the measurement of nucleosomes in a test sample, comprising: providing an immunoassay which comprises a calibrant comprised of a biologically derived nucleosome preparation assigned, or defined by, values expressed in absolute units of mass or concentration.

18. The immunoassay method according to claim 17, wherein said immunoassay comprises a binding agent that binds specifically to nucleosomes.

19. The immunoassay method according to claim 17, wherein the immunoassay employs a single binding agent.

20. The immunoassay method according to claim 17, wherein the immunoassay is a 2-site immunometric assay employing two binding agents.

21. The immunoassay method according to claim 18, wherein the binding agent is directed to a histone, nucleosome core, DNA epitope or a protein adducted to a nucleosome.

22. The immunoassay method according to claim 18, wherein the binding agent is a chromatin protein or an antibody.

23. (canceled)

24. A comparative analytical procedure kit, comprising: a biologically derived nucleosome preparation which contains a defined amount or concentration of nucleosomes expressed in absolute units of mass or concentration, and optionally instructions for use of the kit in a method of diagnosing and/or monitoring disease.

25. (canceled)

26. The method of claim 9, wherein the methylation of a lysine residue is methylation of a histone 3 lysine residue.
